SEQUENCE LISTING (3 sequences)

SEQ ID NO: 1 - DNA, 595 bp, Maize Zea mays L.
aagcttagattacaaggtagtgaattgtgacatgtattcgttcctatccgatccgtcgtttttgagcactaggtgcggtcactgtgacgcgtggacttggcttcgcccactgccatcgtggacccacgtcatcagcaagtgtccatatccaccacccgacccgacgaccgcttgccgtccgatccgtgtgctcccgagggcaaggatggcatttcgccacgcgagatatttttcggtggcctgcacaggccggcagtgcagcggccaaaacgaggtcaggtcagtcacgctgggccccgcctcacgctcccgtcctgctccgggtcccaacaaagccgtccccgggaggtgctcgtgtgctcgtagcgcggtggcgaccccgatgccccgcatattccactgggcgtccgcgccgtcggatgggatcaggacggccgcggcggccccgcgctcggctataaagacgctgcgggggacgcattccctctccgtgctttcttagaggtgggttggcttctcctccccctccggttcgggttcgggttcgtgaggttctccggggttcgggttcgtgggtgagcggatcgagccatgg

SEQ ID NO: 2 - DNA, 1888 bp, Artificial Sequence
  Synthesized
ccatggatggacaacaatcccaacatcaacgagtgcattccctacaactgcctttccaatcccgaggtggaggtgcttggtggtgagaggatcgagaccggttacactcccatcgacatctccctttcccttacccagttccttctttccgagttcgtgcccggtgccggtttcgtgcttggtcttgtggacatcatctggggcatcttcggtccctcccagtgggacgccttccttgtgcagatcgagcagcttatcaaccagaggatcgaggagttcgccaggaaccaggccatctccaggcttgagggtctttccaacctttaccagatctacgccgagtccttcagggagtgggaggccgatcccaccaatcccgcccttagggaggagatgaggatccagttcaacgacatgaactccgcccttaccaccgccatcccactgttcgccgtgcagaactaccaggtgccactgctgtccgtgtacgtgcaggccgccaaccttcacctttccgtgcttagggacgtgtccgtgttcggtcagaggtggggtttcgacgccgccaccatcaactccaggtacaacgaccttaccaggcttatcggtaactacaccgaccacgccgtgaggtggtacaacaccggtcttgagagggtgtggggtcccgactccagggactggatcaggtacaaccagttcaggagggagcttacccttaccgtgcttgacatcgtgtccctgttccctaactacgactccaggacgtaccctatcaggaccgtgtcccagcttaccagggagatctacaccaacccagtgcttgagaacttcgacggttccttccgcggttccgcccagggtatcgaggggtccatcaggagcccacaccttatggacatccttaactccatcaccatctacaccgacgcccaccgcggtgagtactactggtccggccaccagatcatggccagcccagtgggtttctccggtcccgagttcaccttcccactttacggtaccatgggtaacgccgctccacagcagaggatcgtggcccagcttggtcagggtgtgtacaggaccctttcctccaccctttacaggaggcccttcaacatcggtatcaacaaccagcagctttccgtgcttgacggtaccgagttcgcctacggtacctcctccaaccttccctccgccgtgtacaggaagtccggtaccgtggactcccttgacgagattccaccacagaacaacaacgtgccaccaaggcagggtttctcccacaggctttcccacgtgtccatgttcaggtccggtttctccaactcctccgtgtccatcatcagggctccaatgttctcctggatccacaggtccgccgagttcaacaacatcatccccagcagccagatcacccagatccccctgaccaagagcaccaacctgggcagcggcaccagcgtggtgaagggccccggcttcaccggcggcgacatcctgcgccgcaccagccccggccagatcagcaccctgcgcgtgaacatcaccgcccccctgagccagcgctaccgcgtccgcatccgctacgccagcaccaccaacctgcagttccacaccagcatcgacggccgccccatcaaccagggcaacttcagcgccaccatgagcagcggcagcaacctgcagagcggcagcttccgcaccgtgggcttcaccacccccttcaacttcagcaacggcagcagcgtgttcaccctgagcgcccacgtgttcaacagcggcaacgaggtgtacatcgaccgcatcgagttcgtgcccgccgaggtgaccttcgaggccgagtacgacctggagagggctcagaaggccgtgtgaggtcacc

SEQ ID NO: 3 - protein, 624 aa, Artificial Sequence
  Synthesized
MDNNPNINECIPYNCLSNPEVEVLGGERIETGYTPIDISLSLTQFLLSEFVPGAGFVLGLVDIIWGIFGPSQWDAFLVQIEQLINQRIEEFARNQAISRLEGLSNLYQIYAESFREWEADPTNPALREEMRIQFNDMNSALTTAIPLFAVQNYQVPLLSVYVQAANLHLSVLRDVSVFGQRWGFDAATINSRYNDLTRLIGNYTDHAVRWYNTGLERVWGPDSRDWIRYNQFRRELTLTVLDIVSLFPNYDSRTYPIRTVSQLTREIYTNPVLENFDGSFRGSAQGIEGSIRSPHLMDILNSITIYTDAHRGEYYWSGHQIMASPVGFSGPEFTFPLYGTMGNAAPQQRIVAQLGQGVYRTLSSTLYRRPFNIGINNQQLSVLDGTEFAYGTSSNLPSAVYRKSGTVDSLDEIPPQNNNVPPRQGFSHRLSHVSMFRSGFSNSSVSIIRAPMFSWIHRSAEFNNIIPSSQITQIPLTKSTNLGSGTSVVKGPGFTGGDILRRTSPGQISTLRVNITAPLSQRYRVRIRYASTTNLQFHTSIDGRPINQGNFSATMSSGSNLQSGSFRTVGFTTPFNFSNGSSVFTLSAHVFNSGNEVYIDRIEFVPAEVTFEAEYDLERAQKAV

(19) United States
(12) Patent Application Publication
(10) Pub. No.: US 2021/0163973 A1
(43) Pub. Date: Jun. 3, 2021

(54) PLANT CONSTITUTIVE EXPRESSION PROMOTER AND APPLICATIONS THEREOF

(71) Applicant: CHINA AGRICULTURAL UNIVERSITY, Beijing (CN)

(72) Inventors: Jinsheng LAI, Beijing (CN); Haiming ZHAO, Beijing (CN); Weibin SONG, Beijing (CN); Jinjie ZHU, Beijing (CN)

(21) Appl. No.: 16/758,310

(22) PCT Filed: Aug. 14, 2018
(86) PCT No.: PCT/CN2018/100319

§ 371 Date: Feb. 12, 2021

(30) Foreign Application Priority Data
Aug. 16, 2017 (CN) 201710702435.4

(51) Int. Cl. C12N 15/82

(52) U.S. Cl. CPC C12N 15/8222

(57) ABSTRACT

The present invention discloses a method for expressing a target gene and its special specific DNA molecule. The specific DNA molecule provideds in the present invention has a nucleotide sequence as shown at positions 7 to 589 of SEQ ID No.: 1 from the 5′ end in the sequence listing. The experiment proves that the specific DNA molecule provided in the present invention may start the target gene (such as mCrylAb gene, the nucleotide sequence thereof is shown at positions 7 to 1881 from the 5′ end of SEQ ID No.: 2 in the sequence listing) in various tissues of rice, maize and Arabidopsis thaliana, and shows that the specific DNA molecule is a constitutive expression promoter. The invention has important application value.

### TECHNICAL FIELD

The invention relates to the field of plant molecular biology, in particular to a plant constitutive expression promoter and use thereof.

### BACKGROUND OF THE INVENTION

During the development of transgenic plant products, it is needed that protein products are expressed at a high level through transgenic technology. In order to manipulate plants so as to change or improve phenotypic characteristics (such as resistance to biological or abiotic stresses, increased yield, improved quality, etc.), the expression of specific genes in plant tissues is required. This gene manipulation has been made it possible by two discoveries as follows: the ability to transform heterologous genetic materials into plant cells, and the presence of promoters that may drive the expression of heterologous genetic materials.

Promoters are important cis-acting elements that may regulate the transcription of genes. They are divided into three types: constitutive, inducible, and tissue-specific promoters according to the transcription mode of the promoter. Currently, constitutive promoters are widely used and are often used to overexpress specific genes.

The most commonly used promoters include the cauliflower mosaic virus CaMV35S promoter (Odell et. al, Nature 313: 810-812 (1985)), the nopaline synthase (NOS) promoter (Ebert et. al, PNAS. 84: 5745-5749 (1987)), Adh promoter (Walker et. al, PNAS. 84: 6624-6628 (1987)), sucrose synthase promoter (Yang et. al, PNAS. 87: 4144-4148 (1990)) and maize ubiquitin promoter (Cornejo et. al, Plant Mol Biol. 23: 567-581 (1993)). Identifying and isolating regulatory elements that may be used to strongly express specific genes in plants play an important role in the development of commercial varieties of transgenic plants.

### SUMMARY OF THE INVENTION

The technical problem to be solved in the present invention is how to start the expression of the target gene.

To solve the above technical problem, the present invention provides a specific DNA molecule for the first time.

The specific DNA molecule provided in the present invention may be a DNA molecule as shown in a1) or a2) or a3) as follows:

a1) a DNA molecule whose nucleotide sequence is shown at positions 7 to 589 from the 5′ end of SEQ ID No.: 1 in the sequence listing;

a2) a DNA molecule whose nucleotide sequence has an identity of 75% or greater to the nucleotide sequence defined in al) and has a promoter function;

a3) a DNA molecule that hybridizes to the nucleotide sequence defined in a1) or a2) under stringent conditions and has a promoter function.

The expression cassette containing the specific DNA molecule also belongs to the protection scope of the present invention.

The expression cassette (from 5′ to 3′) may include a promoter region (consisting of the specific DNA molecule), a transcription starting region, a target gene region, a transcription termination region, and an optional translation termination region. The promoter region and the target gene region may be natural/similar to the host cell, or the promoter region and the target gene region may be natural/similar to each other, or the promoter region and/or a target gene region are heterologous to the host or to each other. As used in this article, “heterologous” refers to a sequence is derived from a foreign species, or if the sequence is from the same species, the natural form is substantially modified in terms of components or genomic loci through deliberate human intervention. The optionally included transcription termination region may be homologous to the transcription starting region, to the operably linked target gene region, and to the plant host; or the target gene region and host are foreign or heterologous. The transcription termination region may be derived from the Ti-plasmid of Agrobacterium tumefaciens, such as octopine synthase and nopaline synthase termination regions.

The expression cassette may also include a 5′ leader sequence. The 5′ leader sequence may enhance the translation.

In preparing the expression cassette, adapters or linkers may be used to join the DNA fragments, or other operations may be involved to provide appropriate restriction sites, remove excess DNA, remove restriction sites, and so on. To achieve this goal, in vitro mutations, primer repairs, restriction endonuclease digestion, annealing, and replacements, such as switching and transversion, may be conducted.

The expression cassette may also include selective marker genes for screening transformed cells. Selective marker genes may be used to screen transformed cells or tissues. Marker genes include genes encoding antibiotic resistance, such as genes encoding neomycin phosphotransferase II (NEO), hygromycin phosphotransferase (HPT), genes for providing resistance against herbicide compounds (for example, glufosinate, 2,4-D). Other selective markers include phenotypic markers, such as fluorescent proteins. The selective markers listed above are not restrictive. Any selective marker genes may be used in the present invention.

The recombinant plasmid containing the specific DNA molecule also belongs tothe protection scope of the present invention. The recombinant plasmid may be one obtained by inserting the specific DNA molecule into the starting plasmid. The recombinant plasmid may be specifically one obtained by inserting the specific DNA molecule into multiple cloning sites of the starting plasmid.

The starting plasmid may include a selective marker gene for screening transformed cells. Selective marker genes may be used for screening transformed cells or tissues. Marker genes include genes encoding antibiotic resistance, such as genes encoding neomycin phosphotransferase II (NEO), hygromycin phosphotransferase (HPT), genes for providing resistance against herbicide compounds (for example, glufosinate, 2,4-D). Other selective markers include phenotypic markers, such as fluorescent proteins. The selective markers listed above are not restrictive. Any selective marker genes may be used in the present invention.

The recombinant plasmid may include any of the above-mentioned expression cassettes containing the specific DNA molecule.

The recombinant plasmid specifically refers to the recombinant plasmid pCAMBIA3301-Gly. The recombinant plasmid pCAMBIA3301-Gly is obtained by replacing a small fragment between recognition sequences of the restriction endonucleases HindIII and NcoI of the vector pCAMBIA3301 with a DNA molecule having the nucleotide sequence shown at positions 7 to 589 from the 5′ end of SEQ ID No.: 1 in the sequence listing.

Recombinant microorganisms containing the specific DNA molecules also belongs tothe protection scope of the present invention.

The recombinant microorganism may be obtained by introducing the recombinant plasmid into the starting microorganism.

The starting microorganism may be bacteria, yeast, algae or fungi. The bacteria may be gram-positive bacteria or gram-negative bacteria. The gram-negative bacteria may be Agrobacterium tumefaciens. Agrobacterium tumefaciens may be specifically Agrobacterium tumefaciens EHA105 or Agrobacterium tumefaciens GV3101.

The recombinant microorganism may be specifically EHA105/pCAMBIA3301-Gly::mcry1Ab or GV3101/pCAMBIA3301-Gly::mcry1Ab. EHA105/pCAMBIA3301-Gly::mcry1Ab is a recombinant Agrobacterium obtained by transforming the recombinant plasmid pCAMBIA3301-Gly::mcry1Ab into Agrobacterium tumefaciens EHA105. GV3101/pCAMBIA3301-Gly::mcry1Ab is a recombinant Agrobacterium obtained by introducing the recombinant plasmid pCAMBIA3301-Gly::mcry1Ab into Agrobacterium tumefaciens GV3101. The recombinant plasmid pCAMBIA3301-Gly::mcry1Ab may be obtained by replacing a small fragment between recognition sequences of the restriction endonucleases HindIII and NcoI of the vector pCAMBIA3301 with a DNA molecule having the nucleotide sequence shown at positions 7 to 589 from the 5′ end of SEQ ID No.: 1 in the sequence listing, the small fragment between the recognition sequences of the restriction endonucleases NcoI and BstEII with a DNA molecule having the nucleotide sequence shown at positions 7 to 1881 from the 5′ end of SEQ ID No.: 2 in the sequence listing.

Transgenic cell lines containing the specific DNA molecules also belongs to the protection scope of the present invention.

None of the transgenic cell lines containing the specific DNA molecules include propagation materials. It should be understood that the transgenic plant includes not only the first-generation transgenic plants obtained by transforming the specific DNA molecule into a recipient plant, but also progenies of the first-generation transgenic plants. For transgenic plants, the gene may be propagated in this species, or conventional breeding techniques may be used to transfer the gene into other varieties of the same species, specifically including commercial varieties. The transgenic plants include seeds, callus tissues, whole plants and cells.

Use of the specific DNA molecule, the expression cassette or the recombinant plasmid in starting the expression of the target gene also belons tothe protection scope of the present invention.

To solve the above technical problems, the present invention also provides a method for expressing the target gene.

The method for expressing a target gene provided in the present invention may be specifically method I, comprising the step of inserting the specific DNA molecule into the upstream of any target gene or enhancer to start the expression of the target gene.

The method for expressing a target gene provided in the present invention may be specifically method II, comprising the step of inserting the target gene into the downstream of the specific DNA molecule in the expression cassette to start the expression of the target gene by the specific DNA molecule.

The method for expressing a target gene provided in the present invention may be specifically method III, comprising the step of inserting the target gene into the downstream of the specific DNA molecule in the recombinant plasmid to start the expression of the target gene by the specific DNA molecule.

The method for expressing a target gene provided in the present invention may be specifically method IV, in which the specific DNA molecule is used as a promoter or a constitutive promoter to start the expression of the target gene.

In the above, the specific DNA molecule may be used as a promoter (specifically a constitutive promoter) to express a gene (such as a foreign gene) in plants, animals or microorganisms.

Any of the above plants include but are not limited to dicotyledonous and monocotyledonous plants. Examples of related plants include but are not limited to jasmine, brassica, alfalfa, rice, sorghum, foxtail millet (such as millet, Millet, MILLET, finger millet), sunflower, safflower, wheat, soybean, tobacco, potato, peanut, cotton, sweet potato, cassava, coffee, coconut, pineapple, citrus tree, cocoa, tea, banana, avocado, fig, guava, mango, olive, papaya, cashew, macadamia, almond, beet, sugar cane, oatmeal, barley, arabidopsis, vegetables, ornamental plants and conifers. Vegetables may include tomato, lettuce, kidney beans, lima beans, peas, and members of the genus cucumis (for example, cucumber, reticulated melon, and melon). Ornamental plants may include rhododendrons, hydrangeas, hibiscus, roses, tulips, daffodils, petunia hybrida, carnations, poinsettia and chrysanthemums. Conifers that may be used in the practice of the present invention, such as pines (such as loblolly pine, pinus elliottii, pinus ponderosa, pitwood pine, and pinus radiata), douglas fir, tsuga heterophyla, picea sitchensis, sequoia sempervirens, fir (such as European fir and balsam fir), cedar (such as picea sitchensis and nootka cypress). In a specific embodiment, the plant of the invention is a crop (such as maize, rice) or a model plant (such as arabidopsis).

Any of the above-mentioned microorganisms may include bacteria, algae or fungi. Bacteria of particular interest include, for example, Pseudomonas, Erwinia, Serratia, Klebsiella, Flavobacterium, Streptomyces, Rhizobium, Rhodopseudomonas, Methylius, Agrobacterium, Acetobacter, Lactobacillus, Arthrobacter, Azotobacter, Leuconostoc, and Alcaligenes. Fungi include yeast, and those of particular interest are Saccharomyces, Cryptococcus, Kluyveromyces, Sporobolomyces, pichia, and Aureobasidium. Other exemplary prokaryotes (gram-negative or gram-positive), including Enterobacteriaceae (such as E. coli, Erwinia, Shigella, Salmonella, and Proteus), Bacillus, Rhizobia Family (such as Rhizobium), Spirillaceae (such as Photobacterium, Zymomonas, Serratia, Aeromonas), Pseudomonas (such as Pseudomonas and Acetobacter), Azotobacteraceae and Nitrobacteriaceae. Among the eukaryotic cells are fungi, for example, algal fungi and ascomycetes, which include yeasts (such as Saccharomyces and Schizosaccharomyces), Basidiomycetes (such as Pichia, Aureobasidium and Sporobolomyces). The Agrobacterium tumefaciens may be specifically Agrobacterium tumefaciens EHA105 or Agrobacterium tumefaciens GV3101.

Any of the above-mentioned target genes may be the mCry1Ab gene. The nucleotide sequence of the mCry1Ab gene is shown at positions 7 to 1881 from the 5′ end of SEQ ID No.: 2 in the sequence listing.

The experiment proves that the specific DNA molecule provided in the present invention may start the expression of a target gene (such as mCry1Ab gene whose nucleotide sequence is shown at positions 7 to 1881 from the 5′ end of SEQ ID No: 2 in the sequence listing) in various tissues of rice, maize and Arabidopsis thaliana, showing the specific DNA molecule is a constitutive expression promoter. The present invention has important application value.

### BRIEF DESCRIPTION OF THE DRAWINGS

FIG. 1 shows the experimental results of Step 1 of Example 1.

FIG. 2 shows the experimental results of Example 2.

### BEST MODE OF IMPLEMENTING THE INVENTION

The present invention will be described in future detail below withspecific embodiments, and the examples given are only to illustrate the present invention, not to limit the scope of the present invention.

Unless otherwise specified, the experimental methods used in the following examples are conventional methods.

Unless otherwise specified, the materials and reagents used in the following examples are commercially available.

In the following quantitative experiments, three repeated experiments are set, and the results are obtained.

The maize inbred line B73 is from the National Germplasm Resource Bank (website: http://www.cgris.net/), and it is available for the public from China Agricultural University (that is, the applicant) to repeat this experiment. Hereafter, the maize inbred line B73 is abbreviated as B73.

pEASYT1 Cloning Vector and 10×PCR buffer are products of Beijing Quanshijin Biotechnology Co., Ltd. The plasmidpCAMBIA3301 is a product of Huayueyang Biological Technology Co., Ltd., and its catalog number is VECT0150.

Definition of FPKM value: If 1 million reads generated by the second-generation sequencing are mapped to the genome of maize, then how many are mapped to each gene, and since the length of exons is different, then how many reads are mapped to every 1K bases, this is probably the intuitive explanation of the FPKM. FPKM=total exon fragments/(mapped reads (millions)×exon length (kb)).

The solutes and concentration in N6E medium are 4 g/L of N6 salt, 5 mL/L of N6 vitamin Stock (200×), 2 mg/L of 2,4-D, 0.1 g/L of inositol, 2.76 g/L of proline, 30 g/L of sucrose, 0.1 g/L of casein hydrolysate, 2.8 g/L of plant gel and 3.4 mg/L of silver nitrate; the solvent is distilled water; the pH value is 5.8.

N6 vitamin Stock (200×): an aqueous solution containing 0.4 g/L of glycine, 0.1 g/L of nicotinic acid, 0.2 g/L of VB1 and 0.1 g/L of VB6.

N6E solid plate: pour N6E medium at about 55° C. into a petri dish, and cool to obtain an N6E solid plate.

Impregnation medium: 68.4 g of sucrose, 50 mL (large amount of) of N6 (20×), 10 mL (trace) of B5 (100×), 10 mL of N6 iron salt (100×), 5 mL of RTV organic (200×) and 100 μmol of acetosyringone (AS) are dissolved in 1 L of distilled water and the pH is adjusted to 5.2.

Large amount of N6 (20×): an aqueous solution containing 9.26 g/L of (NH₄)₂SO_{4,} 56.60 g/L of KNO₃, 8.00 g/L of KH₂PO₄, 3.70 g/L of MgSO₄.7H₂O and 3.32 g/L of CaCl₂.2H₂O.

B5 trace (100×): an aqueous solution containing 0.7600 g/L of MnSO₄.H₂O, 0.2000 g/L of ZnSO₄.7H₂O, 0.3000 g/L of H₃BO₃, 0.0750 g/L of KI, 0.0250 g/L of Na₂MoO₄.2H₂O, 0.0025 g/L of CuSO₄.5H₂O and 0.0025 g/L of CoCl₂.6H₂O.

N6 iron salt (100×): an aqueous solution containing 1.8300 g/L of sodium iron salt of ethylenediamine tetraacetic acid.

RTV organic (200×): obtained by dissolving 0.0196 g of choline chloride, 0.0098 g of VB₂, 0.0200 g of D-biotin, 0.0400 g of niacin, 0.0097 g of folic acid, 0.0944 g of VB₁, 0.0200 g of calcium D-pantothenate, 0.0400 g of VB₆, 0.0098 g of p-aminobenzoic acid and 400 μL of VB₁₂ aqueous solution with a concentration of 0.75 mg/100 mL in 1 L of distilled water.

Co-culture medium: dissolving 4.33 g of MS salt, 2 mL of MS Vitamins (500×), 0.5 mg of thiamine hydrochloride, 30.0 g of sucrose, 1.38 g of L-proline, 0.5 mg of 2,4-D, 0.01 mg of 6-BA, 3.5 g of plant gel and 100 μmol of AS in 1 L of distilled water, and adjusting the pH to 5.7.

MS Vitamins (500×): an aqueous solution containing 1 g/L of glycine, 0.25 g/L of niacin, 0.05 g/L of VB1 and 0.25 g/L of VB₆.

Recovery medium: dissolving 4.33 g of MS salt, 2 mL of MS Vitamins (500×), 0.5 mg of thiamine hydrochloride, 30.0 g of sucrose, 1.38 g of L-proline, 0.5 mg of 2,4-D, 0.01 mg of 6-BA, 3.5 g of plant gel, 100 mg of Tim, 3.0 mg of dialaphos and 33.4 mg of AgNO in 1 L of distilled water, and adjusting the pH to 5.7.

Primary selection medium: MS solid medium containing 1.5 mg/L of dialaphos.

Secondary selection medium: MS solid medium containing 3.0 mg/L bialaphos.

Regeneration medium I: dissolving 4.33 g of MS salt, 2 mL of MS Vitamins (500×), 0.5 mg of thiamine hydrochloride, 10.0 g of sucrose, 20 g of glucose, 0.7 g of L-proline, 3.5 g of plant gel, 0.2 g of casein hydrolysate, 0.04 g of glycine, 0.1 g of inositol and 3.0 mg of bisphosphinate in 1 L of distilled water, and adjusting the pH to 5.7.

Regeneration medium II: dissolving 2.165 g of MS salt, 30.0 g of sucrose, 3.5 g of plant gel and 3.0 mg of dialanine in 1 L of distilled water, and adjusting the pH to 5.7.

### EXAMPLE 1

### Discovery of Glycine-Rich RNA Binding Protein 2 Promoter

I. Discovery of Glycine-Rich RNA Binding Protein 2 Promoter (Gly Promoter)

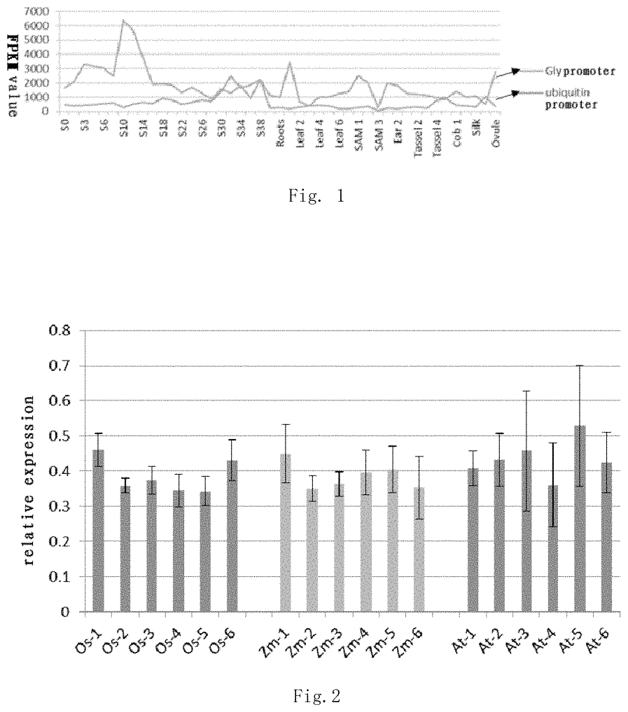

Cell transcriptome analysis is conduction on different tissues of B73 (such as seedlings, roots growing up to 14 days, 1^{st} to 7^{th} leaves, apical meristems at different stages, female spikes at different stages, tassels at different stages, and cobs silk, anthers, ovules at different stages, and grains of different days after B73 self-pollination) by the inventors of the present invention. The results of FPKM values are shown in FIG. 1. The results show that glycine-rich RNA-binding protein 2 gene (gene number Zm00001d031168) is expressed at a high level in all above-mentioned tissues. The gene promoter is referred to as Gly promoter. Compared with the ubiquitin promoter widely used in plants, the expression of Gly promoter is significantly increased in most tissues. Therefore, the application prospect of the Gly promoter is broader.

Comparing the glycine-rich RNA-binding protein 2 gene in maize with genomes of rice, sorghum and Arabidopsis thaliana, homologous genes in rice, sorghum and Arabidopsis thaliana may be identified with gene IDs OS12G0632000, SORBI_001G022600, AT4G13850, respectively. The 600 bp sequences upstream of the transcription start site of these genes have the same function as the Gly promoter sequence reported in the present invention.

II. Cloning of the Gly Promoter

1. Genomic DNA of leaves of B73 is extracted and used as a template, using a primer pair consisting of primer 1: 5′-AAGCTTAGATTACAAGGTAGTGAATTGTGACATG-3′ (underlined for the recognition site of restriction endonuclease HindIII) and primer 2: 5′-CCATGGCTCGATCCGCTCACCCACG-3′ (underlined for the recognition site of restriction endonuclease NcoI) to perform PCR amplification to obtain PCR amplification products.

The reaction system is 20 μL, consisting of 2 μL of 10×PCR buffer, 1.6 μL of dNTP with a concentration of 10 mM (that is, the concentration of all of dATP, dTTP, dCTP, and dGTP is 10 mM), 0.5 μL of aqueous solution of primer 1, 0.5 μL of aqueous solution of primer 2, 2 μL of the template, 0.3 μL of Taq enzyme and 13.1 μL of ddH2O. In the reaction system, the concentration of both primer 1 and primer 2 is 10 nM, and the concentration of template is 10-100 ng/μL.

Reaction conditions: pre-denaturation at 94° C. for 6 min; 34 cycles of denaturation at 94° C. for 30 s, annealing at 58° C. for 30 s, extension at 72° C. for 30 s; and extension at 72° C. for 10 min.

2. After step 1 is completed, the PCR amplification product is detected by 2% (2 g/100 mL) agarose gel electrophoresis, and then the PCR amplification product of about 595 bp is recovered.

3. After step 2 is completed, connect the PCR amplification product of about 595 bp with pEASYT1 cloning vector to obtain the recombinant plasmid pEASYT1-GlyP.

The recombinant plasmid pEASYT1-GlyP is sequenced. The sequencing results show that the recombinant plasmid pEASYT1-GlyP contains the DNA molecule shown in SEQ ID No.: 1 in the sequence listing. In the sequence listing, the DNA molecule shown at positions 7 to 589 from the 5′end in SEQ ID No.: 1 of the sequence listing is the nucleotide sequence of the Gly promoter.

### EXAMPLE 2

### Use of Gly Promoter in the Expression of Mcry1Ab Gene

I. Construction of Recombinant Plasmid pCAMBIA3301-Gly::mcry1Ab

1. Double-digest the recombinant plasmid pEASYT1-GlyP with restriction endonucleases HindIII and NcoI to recover DNA fragment 1 of about 580 bp.

2. Double-digest the vector pCAMBIA3301 with restriction endonucleases HindIII and NcoI, and recover the vector backbone 1 of about 10 kb.

3. Connect the DNA fragment 1 to the vector backbone 1 to obtain the recombinant plasmid pCAMBIA3301-Gly.

4. The double-stranded DNA molecule shown in SEQ ID No.: 2 in the sequence listing is artificially synthesized, and then double-digested with restriction endonucleases NcoI and BstEII, and a DNA fragment 2 of about 1.9 kb is recovered. In the sequence listing, the DNA molecule shown at positions 7 to 1881 from the 5′end of SEQ ID No.: 2 is a gene encoding mCry1Ab protein (hereinafter referred to as mCry1Ab gene). The amino acid sequence of mCry1Ab protein is shown as SEQ ID No.: 3 in the sequence listing.

5. Double-digest the recombinant plasmid pCAMBIA3301-Gly with restriction endonucleases NcoI and BstEII to recover the vector backbone 2 of about 10 kb.

6. Connect the DNA fragment 2 and the vector backbone 2 to obtain the recombinant plasmid pCAMBIA3301-Gly::mcry1Ab.

The recombinant plasmid pCAMBIA3301-Gly::mcry1Ab is sequenced. According to the sequencing results, the recombinant plasmid pCAMBIA3301-Gly::mcry1Ab was structurally described as follows: the small fragment between recognition sequences of the restriction endonucleases HindIII and NcoI of the vector pCAMBIA3301 is replaced with a DNA molecule whose nucleotide sequence is shown at positions 7 to 589 from the 5′ end of SEQ ID No.: 1 in the sequence listing, the small fragment between the recognition sequences of the restriction endonucleases NcoI and BstEII is replaced with a DNA molecule whose nucleotide sequence is shown at positions 7 to 1881 from the 5′ end of SEQ ID No.: 2 in the sequence listing. The recombinant plasmid pCAMBIA3301-Gly::mcry1Ab expresses the mCry1Ab protein shown in SEQ ID No.: 3 in the sequence listing.

II. Obtaining Transgenic Rice Transformed with mCry1Ab Gene and Functionally Verifying Gly Promoter

1. Obtaining Recombinant Agrobacterium

The recombinant plasmid pCAMBIA3301-Gly::mcry1Ab is introduced into Agrobacterium tumefaciens EHA105 to obtain a recombinant Agrobacterium, which is named EHA105/pCAMBIA3301-Gly::mcry1Ab.

The recombinant plasmid pCAMBIA3301 is introduced into Agrobacterium tumefaciens EHA105 to obtain recombinant Agrobacterium, which is named EHA105/pCAMBIA3301.

2. Obtaining mCry1Ab Transgenic Rice

Using the method of Hiei et al. (Hiei Y, Ohta S, Komari T & Kumashiro T. Efficient transformation of rice (Oryza sativa L.) mediated by Agrobacterium and sequence analysis of the boundaries of the T-DNA. Plant J. 1994, 6: 271-282), EHA105/pCAMBIA3301-Gly::mcry1Ab is transformed into the rice variety Nipponbare to obtain rice transformed with mCry1Ab gene. Five of the rice transformed with mCry1Ab genes are named Os-1 to Os-5, respectively.

According to the above method, EHA105/pCAMBIA3301-Gly::mcry1Ab is replaced with EHA105/pCAMBIA3301, and the other steps are the same as the above to obtain an empty vector transgenic rice.

3. Molecular Identification

Genomic DNA of Os-1 to Os-5 leaves is extracted and used as a template, using a pair of primer consisting of primer F4: 5′-TCCGTGCTTTCTTAGAGGTGGGTT-3′ and primer R4: 5′-GAACTCGGAAAGAAGGAACTGGGTAA-3′ for PCR amplification to obtain PCR amplification products.

The reaction system is 20 μL, consisting of 2 μL of 10×PCR buffer, 1.6 μL of dNTP with a concentration of 10 mM (that is, the concentration of all of dATP, dTTP, dCTP, and dGTP is 10 mM), 0.5 μL of aqueous solution of primer F4, 0.5 μL of aqueous solution of primer R4, 2 μL of the template, 0.3 μL of Taq enzyme and 13.1 μL, of ddH₂O. In the reaction system, the concentration of both primer F4 and primer R4 is 10 nM, and the concentration of template is 10-100 ng/μL.

Reaction conditions: pre-denaturation at 94° C. for 10 min; 34 cycles of denaturation at 94° C. for 30 s, annealing at 59° C. for 30 s, extension at 72° C. for 1 min; extension at 72° C. for 10 min.

According to the above method, the genomic DNA of the Os-1 leaf is replaced with water, and the other steps are the same as the above to obtain a negative control.

According to the above method, the genomic DNA of leaves of Os-1 is replaced with the genomic DNA of leaves of the transgenic rice with an empty vector, and the other steps are the same as the above to obtain a control 1.

According to the above method, the genomic DNA of leaves of Os-1 is replaced with the genomic DNA of leaves of the rice variety Nipponbare, and the other steps are the same as the above to obtain a control 2.

According to the above method, the genomic DNA of the Os-1 leaf is replaced with the recombinant plasmid pCAMBIA3301-Gly::mcry1Ab, and the other steps are the same as the above to obtain a positive control.

The PCR amplification products are subjected to agarose gel electrophoresis. The results show that the genomic DNA of Os-1 to Os-5 leaves or the recombinant plasmid pCAMBIA3301-Gly::mcry1Ab may be amplified as a template to obtain a band of 258 bp; when water, the genomic DNA of rice leaves transformed with an empty vector or the genomic DNA of leaves of the rice variety Nipponbare is used as a template, no band of 258 bp could be obtained after amplifying.

According to molecular identification, all of Os-1 to Os-5 are transgenic rice transformed with the mCry1Ab gene.

4. Real-Time Quantitative PCR Detection

The rice to be tested is Os-1, Os-2, Os-3, Os-4, Os-5, transgenic rice transformed with an empty vector or rice variety Nipponbare.

The tissues to be tested are leaves, roots, stalks, flowers, or grains.

(1) Extract the total RNA of the tissue of the rice to be tested, and then perform reverse transcription to obtain the cDNA of the rice to be tested. The DNA content in the cDNA of rice to be tested is about 50 ng/μL.

(2) Detect the relative expression of the mcry1Ab gene in the cDNA of rice to be tested using fluorescence quantitative PCR (the actin gene is used as an internal reference gene).

The primers for detecting the mcry1Ab gene are forward primer 1: 5′-GTGGAGGTGCTTGGTGGTGAGA-3′ and reverse primer 1: 5′-ACTGGGAGGGACCGAAGATGC-3′. The primers for detecting the actin gene are forward primer 2: 5′-GAAGATCACTGCCTTGCTCC-3′ and reverse primer 2: 5′-CGATAACAGCTCCTCTTGGC-3′.

The reaction system is 25 μL consisting of 2 μL of cDNA of rice to be tested, 1 μL of aqueous solution of the forward primer, 1 μL of aqueous solution of the reverse primer, 13 μL of SYBR (product of TAKARA) and 8 μL of ddH₂O. In the reaction system, the concentration of both forward primer and the reverse primer is 10 nM.

Reaction procedure: pre-denaturation at 95° C. for 5 min; 40 cycles of denaturation at 95° C. for 15 s, annealing at 60° C. for 35 s; extension at 72° C. for 5 min; storage at 4° C.

Statistically analyze the relative expression of the mcry1Ab gene in cDNA of the rice to be tested. The experimental results are shown in FIG. 2. The results show that the relative expression levels of the mcry1Ab gene in the tissues of Os-1, Os-2, Os-3, Os-4 and Os-5 are significantly increased compared with the rice variety Nipponbare, and there is no significant difference in the relative expression of the mcry1Ab gene between tissues of transgenic rice with an empty vector and the rice variety Nipponbare.

The above results indicate that the Gly promoter may promote the expression of the mCry1Ab gene in various tissues of rice.

3. Obtaining Transgenic Maize Transformed with mCry1Ab Gene and Functionally Verifying the Promoter

1. Obtaining Recombinant Agrobacterium

The recombinant plasmid pCAMBIA3301-Gly::mcry1Ab is introduced into Agrobacterium tumefaciens EHA105 to obtain a recombinant Agrobacterium, which is named EHA105/pCAMBIA3301-Gly::mcry1Ab.

The recombinant plasmid pCAMBIA3301 is introduced into Agrobacterium tumefaciens EHA105 to obtain recombinant Agrobacterium, which is named EHA105/pCAMBIA3301.

2. Obtaining Transgenic Maize Transformed with the mCry1Ab Gene

(1) Obtaining and Cultivating Immature Embryos

(a) Plant the maize variety X178 in the field, and after 9-11 days of self-pollination, remove the bract leaves of the pollinated ears, and then put the ears in a beaker containing a disinfectant solution (obtained by adding a droplet of Tween 20 to 700 mL of 50% (v/v) bleach solution or a solution of sodium hypochlorite aqueous solution (effective chlorine is 5.25% (v/v)) to soak for 20 min, and then wash with sterile water 3 times. During the soaking process, it is necessary to rotate the ears from time to time while gently tapping the beaker to expel the air bubbles on the surface of the particles to achieve the best disinfection effect.

(b) After the step (a) is completed, take the ears, insert the tip of the embryo peeling knife between the embryo and the endosperm, then gently pry up the immature embryo, and gently lift the immature embryo with a small scalpel tip. To ensure that the immature embryo is not damaged, put the embryonic axis of the immature embryo close to the N6E solid plate with filter paper. The density of the immature embryo is about 2 cm×2 cm (30/dish).

(c) After the step (b) is completed, take the N6E solid plate, seal with parafilm, and incubate at 28° C. for 2-3 days in the dark.

(2) Obtaining Agrobacterium Impregnation Solution

(a) Inoculate EHA105/pCAMBIA3301-Gly::mcry1Ab on YEP solid medium containing 33 mg/L of Kanamycin (Kana) and 50 mg/L of streptomycin (str), and cultivate at 19° C. for 3 days to active.

(b) The EHA105/pCAMBIA3301-Gly::mcry1Ab obtained in step (a) is inoculated in the inoculation medium, and cultured with shaking at 25° C. and 75 rpm to obtain an Agrobacterium impregnation solution with an OD₅₅₀ₙₘ of 0.3-0.4.

(3) Obtaining Transgenic Maize Transformed with the mCry1Ab Gene

The conditions of alternating light and dark culture (that is, alternating light culture and dark culture) are 25° C. The light intensity during the light culture is 15000 Lx. The cycle of alternating light-dark culture is specifically 16 h light culture/8 h dark culture.

(a) Take the immature embryos after step (2) is completed, and place them in a centrifuge tube, wash them twice with impregnation medium (2 mL of impregnation medium is used for each washing), then add Agrobacterium impregnation solution, and gently invert the centrifuge tube for 20 times, and let stand upright for 5 min (make sure all embryos are immersed in the Agrobacterium impregnation solution).

(b) After step (a) is completed, transfer the immature embryos to the co-culture medium (make the embryonic axis of the immature embryos contact the surface of the co-culture medium while removing the excess Agrobacterium on the surface of the co-culture medium), then cultivate at 20° C. in the dark for 3 days.

(c) After step (b) is completed, transfer the immature embryos to the recovery medium, and then culture at 28° C. in the dark for 7 days.

(d) After step (c) is completed, transfer the immature embryos to the primary selection medium, and then incubate in alternating light and dark at 28° C. for two weeks.

(e) After step (d) is completed, transfer the immature embryos to the secondary selection medium, and then culture in alternating light and dark at 28° C. for two weeks to obtain resistant callus.

(f) After step (e) is completed, transfer the resistant callus to regeneration medium I, and then incubate in alternating light and dark at 28° C. for three weeks.

(g) After step (f) is completed, transfer the resistant callus to regeneration medium II, and then incubate in alternating light and dark at 28° C. for three weeks to obtain regenerated seedlings. When the regenerated seedlings grow to 3-4 leaves, they are transferred to the greenhouse and cultivated normally to obtain transgenic maize with mCry1Ab gene. Five of these transgenic maize transformed with the mCry1Ab gene are named Zm-1 to Zm-5, respectively.

According to the above method, EHA105/pCAMBIA3301-Gly::mcry1Ab is replaced with EHA105/pCAMBIA3301, the other steps are the same as the above, a maize transformed with an empty plasmid is obtained.

3. Molecular Identification

Genomic DNA of leaves of Zm-1 to Zm-5 is extracted respectively and used as a template, using a primer pair consisting of primer F4: 5′-TCCGTGCTTTCTTAGAGGTGGGTT-3′ and primer R4: 5′-GAACTCGGAAAGAAGGAACTGGGTAA-3′ for PCR amplification to obtain PCR amplification products.

The reaction system is the same as that in step II (3).

The reaction conditions are the same as those in step II (3).

According to the above method, the genomic DNA of leaves of Zm-1 is replaced with water, and the other steps are the same as the above to obtain a negative control.

According to the above method, the genomic DNA of leaves of Zm-1 is replaced with the genomic DNA of leaves of the transgenic maize with an empty vector, and the other steps are the same as the above to obtain a control 1.

According to the above method, the genomic DNA of leaves of Zm-1 is replaced with the genomic DNA of leaves of maize variety X178, and the other steps are the same as the above to obtain a control 2.

According to the above method, the genomic DNA of leaves of Zm-1 is replaced with the recombinant plasmid pCAMBIA3301-Gly::mcry1Ab, and the other steps are the same as the above to obtain a positive control.

The PCR amplified products are subjected to agarose gel electrophoresis. The results show that the genomic DNA of leaves of Zm-1 to Zm-5 or the recombinant plasmid pCAMBIA3301-Gly::mcry1Ab may be amplified as a template to obtain a band of 258 bp; when water, the genomic DNA of leaves of transgenic maize transformed with an empty vector or the genomic DNA of leaves of maize variety X178 is used as a template, no band of 258 bp could be obtain after amplifying.

According to molecular identification, all Zm-1 to Zm-5 are transgenic maize transformed with mCry1Ab gene.

4. Real-Time Quantitative PCR Detection

The maize to be tested is Zm-1, Zm-2, Zm-3, Zm-4, Zm-5, transgenic maize transformed with an empty vector or maize variety X178.

The tissues to be tested are leaves, roots, female spikes, tassels, cobs, filaments, anthers, ovules or grains.

1. Extract the total RNA of the test tissues of the maize to be tested, and then perform reverse transcription to obtain the cDNA of the maize to be tested. The DNA content in the cDNA of maize to be tested is about 200 ng/μL.

2. Detect the relative expression of the mcry1Ab gene in the cDNA of maize to be tested using fluorescence quantitative PCR (the zssIIb gene is used as an internal reference gene).

The primers for detecting the mcry1Ab gene are the same as those for detecting the mcry1Ab gene in step II (4).

The reaction system is the same as that in step II (4).

The reaction procedure is the same as that in step II (4).

Statistically analyze the relative expression of the mcry1Ab gene in cDNA of maize to be tested. The experimental results are shown in FIG. 2. The results show that the relative expression of the mcry1Ab gene in the tissues of Zm-1, Zm-2, Zm-3, Zm-4 and Zm-5 is significantly increased compared with the maize variety X178, and there is no significant difference in the relative expression of the mcry1Ab gene between tissues of the transgenic maize transformed with an empty vector and the maize variety X178.

The above results indicate that the Gly promoter may promote the expression of the mCry1Ab gene in various tissues of maize.

IV. Obtaining Transgenic Arabidopsis thaliana Transformed with the mCry1Ab Gene and Functionally Verifying Gly Promoter

Colombian ecotype Arabidopsis is a product of the Arabidopsis Biological Resource Center (website: http://abrc.osu.edu/). Hereafter, Colombian ecotype Arabidopsis is referred to as wild-type Arabidopsis for short.

1. Obtaining Recombinant Agrobacterium

The recombinant plasmid pCAMBIA3301-Gly::mcry1Ab is introduced into Agrobacterium tumefaciens GV3101 to obtain a recombinant Agrobacterium, which is named GV3101/pCAMBIA3301-Gly::mcry1Ab.

The recombinant plasmid pCAMBIA3301 is introduced into Agrobacterium tumefaciens GV3101 to obtain a recombinant Agrobacterium, which is named GV3101/pCAMBIA3301.

2. Obtaining Transgenic Arabidopsis thaliana Transformed with mCry1Ab Gene

(1) The Arabidopsis thaliana inflorescence dipping transformation method (described in the following reference: Clough, S J, and Bent, A F. Floral dip: a simplified method for Agrobacterium-mediated transformation of Arabidopsis thaliana. Plant J. (1998) 16, 735-743.) is used, GV3101/pCAMBIA3301-Gly::mcry1Ab is transformed into wild-type Arabidopsis thaliana to obtain T₁ generation of seeds of wild-type Arabidopsis thaliana transformed with the mcry1Ab gene.

2. Seed T₁ generation of seeds of wild-type Arabidopsis thaliana transformed with the mcry1Ab gene on MS medium containing 50 mg/L of Basta. The Arabidopsis thaliana (resistant seedling) that may grow normally is T₁ generation of positive seedling transformed with the mcry1Ab gene. The seeds received from the T₁ generation of positive seedling transformed with the mcry1Ab gene are the T₂ generation of seeds of wild-type Arabidopsis thaliana transformed with mcry1Ab gene.

3. Seed different strains of T₂ generation of seeds of wild-type Arabidopsis thaliana transformed with the mcry1Ab gene on the MS medium containing 50 mg/L Basta for screening. As for a certain strain, if the number of Arabidopsis thaliana that may grow normally (resistant seedlings) to the number of Arabidopsis that may not grow normally (non-resistant seedlings) is 3:1, this strain is one into which a copy of the mcry1Ab gene is inserted. The seeds obtained from the resistant seedlings in this strain are T₃ generation of seeds of wild-type Arabidopsis thaliana transformed with the mcry1Ab gene.

4. Seed T₃ generation of seeds of wild-type Arabidopsis thaliana transformed with the mcry1Ab gene again on the MS medium containing 50 mg/L of Basta for screening, and those that are all resistant seedlings are the T₃ generation of homozygous wild-type Arabidopsis thaliana transformed with the mcry1Ab gene. Five of T₃ generation of homozygous wild-type Arabidopsis thaliana strains are named At-1 to At-5, respectively.

According to the above method, GV3101/pCAMBIA3301-Gly::mcry1Ab is replaced with GV3101/pCAMBIA3301, and the other steps are the same as the above, to obtain T₃ generation of homozygous wild-type Arabidopsis thaliana transformed with an empty vector, referred to as Arabidopsis thaliana transformed with an empty vector.

3. Molecular Identification

Genomic DNA of At-1 to At-5 leaves is extracted and used as a template, using a primer pair consisting of primer F4: 5′-TCCGTGCTTTCTTAGAGGTGGGTT-3′ and primer R4: 5′-GAACTCGGAAAGAAGGAACTGGGTAA-3′ for PCR amplification to obtain PCR amplification products.

The reaction system is the same as that in step II (3).

The reaction conditions are the same as those in step II (3).

According to the above method, the genomic DNA of leaves of At-1 is replaced with water, and the other steps are the same as the above to obtain a negative control.

According to the above method, the genomic DNA of leaves of At-1 is replaced with the genomic DNA of leaves of the Arabidopsis thaliana, and the other steps are the same as the above to obtain a control 1.

According to the above method, the genomic DNA of leaves of At-1 is replaced with the genomic DNA of leaves of wild-type Arabidopsis thaliana, and the other steps are the same as the above to obtain a control 2.

According to the above method, the genomic DNA of the At-1 leaves is replaced with the recombinant plasmid pCAMBIA3301-Gly::mcry1Ab, and the other steps are the same as the above to obtain a positive control.

The PCR amplified products are subjected to agarose gel electrophoresis. The results show that the genomic DNA of At-1 to At-5 leaves or recombinant plasmid pCAMBIA3301-Gly::mcry1Ab may be amplified as a template to obtain a band of 258 bp; when water, the genomic DNA of leaves of Arabidopsis thaliana transformed with an empty vector, or the genomic DNA of leaves of wild-type Arabidopsis thaliana is used as a template, no band of 258 bp could be obtained after amplifying.

According to molecular identification, all At-1 to At-5 are transgenic Arabidopsis thaliana transformed with the mCry1Ab gene.

4. Real-Time Quantitative PCR Detection

The Arabidopsis thaliana to be tested is At-1, At-2, At-3, At-4, At-5, Arabidopsis thaliana transformed with an empty vector or wild-type Arabidopsis thaliana.

The tissues to be tested are leaves, roots, stems or grains.

1. Extract the total RNA of the tissue of the Arabidopsis thaliana to be tested, and then perform reverse transcription to obtain the cDNA of the Arabidopsis thaliana to be tested. The DNA content in the cDNA of Arabidopsis thaliana to be tested is about 200 ng/μL.

2. Detect the relative expression of the mcry1Ab gene in the cDNA of Arabidopsis thaliana to be tested using fluorescence quantitative PCR (the actin gene is used as an internal reference gene).

The primers for detecting the mcry1Ab gene are the same as those for detecting the mcry1Ab gene in step II (4).

The primers for detecting the actin gene are the same as those for detecting the actin gene in step II (4).

The reaction system is the same as that in step II (4).

The reaction procedure is the same as that in step II (4).

Statistically analyze the relative expression of the mcry1Ab gene in the cDNA of Arabidopsis thaliana to be tested. The experimental results are shown in FIG. 2. The results show that the relative expression levels of the mcry1Ab gene in At-1, At-2, At-3, At-4 and At-5 tissues are significantly increased compared with wild-type Arabidopsis thaliana, and there was no significant difference in the relative expression of mcry1Ab gene between tissues of the transgenic Arabidopsis thaliana transformed with an empty vector and the wild-type Arabidopsis thaliana.

The above results indicate that the Gly promoter may start the expression of the mCry1Ab gene in various tissues of Arabidopsis thaliana.

### INDUSTRIAL APPLICATIONS

Introduce the recombinant plasmid pCAMBIA3301-Gly::mcry1Ab (that is, a recombinant plasmid containing the Gly promoter and the mCry1Ab gene) into the starting plants (such as rice, maize, and Arabidopsis) to obtain transgenic plants; after testing, the Gly promoter may start the expression of the mCry1Ab gene in various tissues of the transgenic plants. Therefore, the Gly promoter is a constitutive expression promoter and has important application value.

1. A specific DNA molecule, the specific DNA molecule is a DNA molecule shown in a1) or a2) or a3) as follows:
a1) a DNA molecule whose nucleotide sequence is shown at positions 7 to 589 from the 5′ end of SEQ ID No.: 1 in the sequence listing;
a2) a DNA molecule whose nucleotide sequence has an identity of 75% or greater to the nucleotide sequence defined in a1) and has a promoter function;
a3) a DNA molecule that hybridizes to the nucleotide sequence defined in a1) or a2) under stringent conditions and has a promoter function.

2. An expression cassette containing the specific DNA molecule of claim 1.

3. A recombinant plasmid containing the specific DNA molecule of claim 1.

4. The recombinant plasmid of claim 3, wherein the recombinant plasmid is obtained by inserting the specific DNA molecule into a starting plasmid.

5. The recombinant plasmid of claim 3, wherein the recombinant plasmid is a recombinant plasmid pCAMBIA3301-Gly; the recombinant plasmid pCAMBIA3301-Gly is obtained by replacing a small fragment between recognition sequences of the restriction endonucleases HindIII and NcoI of the vector pCAMBIA3301 with the DNA molecule shown at positions 7 to 589 of SEQ ID No.: 1 from the 5′ end of SEQ ID No.: 1 in the sequence listing.

6. A recombinant microorganism containing the specific DNA molecule of claim

7. The recombinant microorganism of claim 6, wherein the recombinant microorganism is obtained by introducing a recombinant plasmid ontaining the specific DNA molecule into a starting microorganism.

8. The recombinant microorganism of claim 7, wherein the starting microorganism is bacteria, yeast, algae or fungi.

9. A transgenic plant cell line containing the specific DNA molecule of claim 1.

10. Use of the specific DNA molecule of claim 1, in starting expression of a target gene.

11. A method for expressing a target gene, comprising the steps of inserting the specific DNA molecule of claim 1 into the upstream of any target gene or enhancer to thereby start the expression of the target gene.

12. A method of expressing a target gene, comprising the steps of inserting the target gene into the downstream of the specific DNA molecule in the expression cassette of claim 2 to start the expression of the target gene by the specific DNA molecule.

13. A method for expressing a target gene, comprising the steps of inserting the target gene into the downstream of the specific DNA molecule in the recombinant plasmid of claim 3 to start the expression of the target gene by the specific DNA molecule.

14. A method for expressing a target gene, wherein the expression of a target gene is started by the specific DNA molecule of claim 1 as a promoter or a constitutive promoter.

15. The use of claim 10, wherein the target gene is the mCry1Ab gene.
